(19) United States
(12) Patent Application Publication
(10) Pub. No.: US 2019/0175930 A1
(43) Pub. Date: Jun. 13, 2019

(54) TREATMENT OF NAILBED AND OTHER HARD-TO-ACCESS INFECTIONS

(71) Applicant: Weinberg Medical Physics, Inc., North Bethesda, MD (US)

(72) Inventor: Irving N. WEINBERG, North Bethesda, MD (US)

(21) Appl. No.: 16/213,184

(22) Filed: Dec. 7, 2018

Related U.S. Application Data
(60) Provisional application No. 62/596,347, filed on Dec. 8, 2017.

(51) Int. Cl. A61N 2/00; A61B 34/20; A61N 2/02

(52) U.S. Cl. CPC A61N 2/002; A61B 34/20; A61B 5/055; A61B 2034/2055; A61B 2034/2051; A61N 2/02

(57) ABSTRACT

Disclosed embodiments provide a tool and methodologies for delivery and manipulation of the one or more magnetic particles to the subject's body part.

### CROSS REFERENCE AND PRIORITY CLAIM

This patent application claims priority to U.S. Provisional Application Provisional Patent Application No. Patent Application Ser. No. 62/596,347, entitled “TREATMENT OF NAILBED AND OTHER HARD-TO-ACCESS INFECTIONS,” filed Dec. 8, 2017, the disclosure of which being incorporated herein by reference in its entirety.

### FIELD

Disclosed embodiments provide a tool for treatment of nailbed and other hard-to-access infections in a subject's body.

### BACKGROUND

Chronic infections of the nailbed, especially by fungi (e.g., onychomycosis), are often difficult to treat. Such infections are widespread, affecting about 10% of the USA population. Topical medications are often unsuccessful, and even oral medications are 76% effective at best, with recurrence of symptoms being quite common.

### SUMMARY

The following presents a simplified summary in order to provide a basic understanding of some aspects of various invention embodiments. The summary is not an extensive overview of the invention. It is neither intended to identify key or critical elements of the invention nor to delineate the scope of the invention. The following summary merely presents some concepts of the invention in a simplified form as a prelude to the more detailed description below.

Disclosed embodiments provide a tool for treatment of nailbed and other hard-to-access infections in a subject's body.

Disclosed embodiments provide a mechanism for the delivery and manipulation of a plurality of magnetic particles to one or more hard-to-access infection sites within a subject's body.

In accordance with disclosed embodiments, at least one image-guidance component located in proximity to the subject's body may be used to direct, transport, concentrate and/or focus one or more particles within the one or more body locations within a subject's body.

### BRIEF DESCRIPTION OF FIGURES

Further advantages, features and possibilities of using the present disclosed embodiments emerge from the description below in conjunction with the figures.

FIG. 1 illustrates an example of the difficulties in treating nailbed and other hard-to-access infections with a subject's body.

FIG. 2 illustrates an example of a disclosed embodiment for treating nailbed or other hard-to-access infections.

### DETAILED DESCRIPTION

The description of specific embodiments is not intended to be limiting of the present invention. To the contrary, those skilled in the art should appreciate that there are numerous variations and equivalents that may be employed without departing from the scope of the present invention. Those equivalents and variations are intended to be encompassed by the present invention.

In the following description of various invention embodiments, reference is made to the accompanying drawings, which form a part hereof, and in which is shown, by way of illustration, various embodiments in which the invention may be practiced. It is to be understood that other embodiments may be utilized and structural and functional modifications may be made without departing from the scope and spirit of the present invention.

Evidence that magnetic manipulation of magnetic particles can increase penetration into tissues was provided in a presentation and publication-in-press (AIP Advances Journal) by S Jafari, entitled “Magnetically Targeted Delivery Through Cartilage,” at the 2017 Annual Conference on Magnetism and Magnetic Materials (MMM) in Pittsburgh (incorporated herein by reference in its entirety).

For the purposes of this disclosure, it is postulated that the manipulation of particles during application to the nailbed would reduce the pain and discomfort associated with the administration as compared to other methods of introducing medications into the nailbed. For example, the particles could be rotated during insertion and/or during the biofilm disruption process, using features on the particles as described by L. O. Mair in the presentation entitled “Doing the Twist: Nanorods with Orthogonally Magnetized Segments Twist Their Way Through Viscoelastic Media” (incorporated herein by reference in its entirety), at the Scientific and Clinical Applications of Magnetic Carriers Meeting, June 2016, Vancouver, Canada and in the US patent application 2016/0125994, entitled “Method and Apparatus for Non-contact Axial Propulsion and Decoupled Particle Propulsion” (incorporated herein by reference in its entirety).

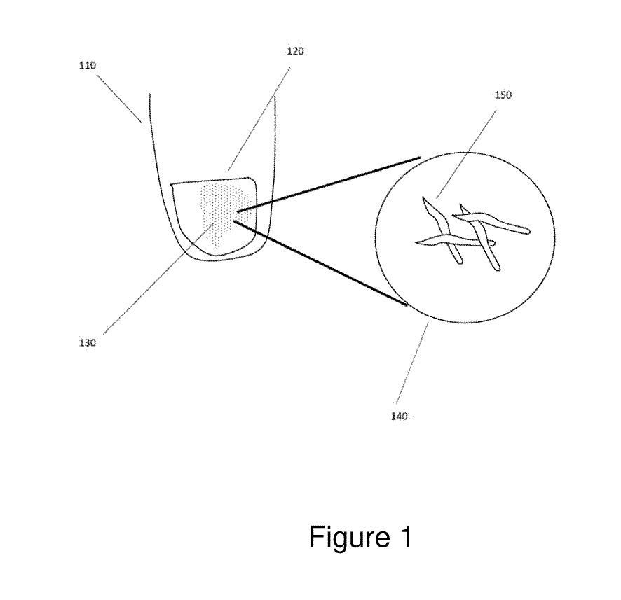

FIG. 1 illustrates an example of the difficulties in treating nailbed and other hard-to-access infections with a subject's body. In FIG. 1, a body part of a human or animal patient (The body part shown as a toe 110 with a toenail 120) contains microbes (for example fungi) 130. Inset 140 shows a magnified view of fungi 150 that are matted into a biofilm.

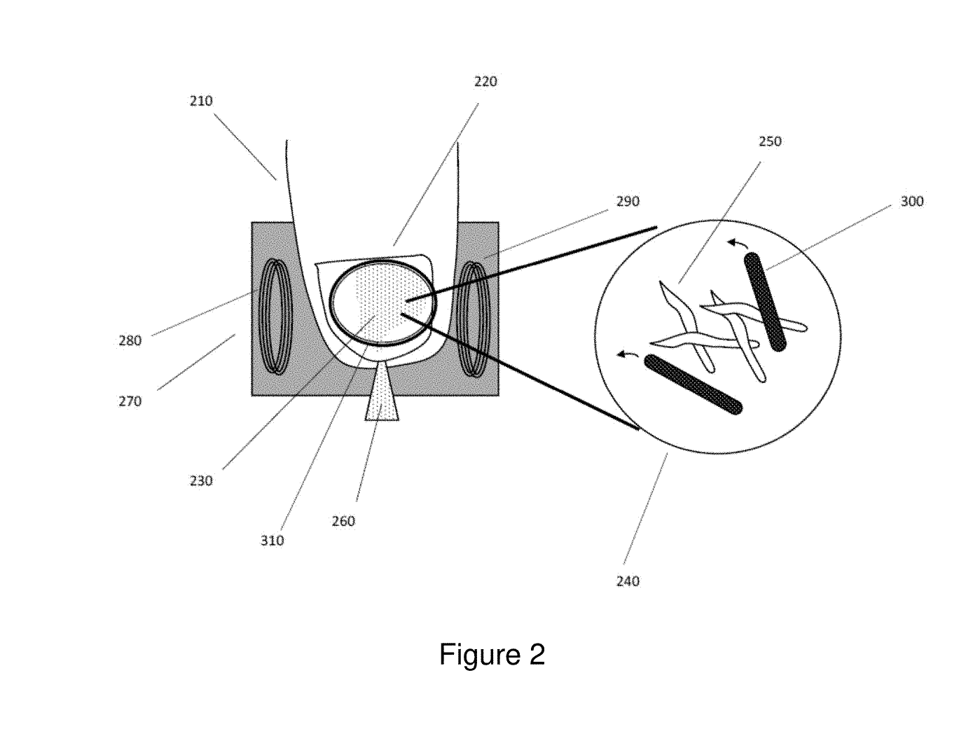

FIG. 2 illustrates an example of a disclosed embodiment for treating nailbed or other hard-to-access infections. FIG. 2 again shows the anatomy of the toe 210 with a toenail 220, again with an infection 230, which is shown in an inset to contain fungi 250. In FIG. 2, particles 300 as in the disclosed apparatus have been introduced into the subject's body. In accordance with disclosed embodiments, an applicator 260 is used to insert particles 300 into the toenail bed of the subject.

Thus, as shown in FIG. 2, an apparatus is provided for increasing the effectiveness of treatment to infections of the nail bed under a toenail 220. The apparatus includes an applicator 260 for local administration of magnetic particles 300 into the nail bed, as well as a magnetic field generator 270 (including coils and/or permanent magnets 280 and 290) for manipulating magnetic particles 300. The apparatus may further include a mechanism 310 for imaging the nailbed and particles 300, the mechanism being optical or other modalities (for example, magnetic resonance imaging, abbreviated as MRI). As shown in FIG. 2, the inset 240 shows the microbes 250, now accompanied by introduced magnetic particles 300, where the particles 300 are moved in response to a magnetic field created by magnetic field generator 270.

An optical objective lens 310 is shown which represents a component, or components, that monitors the insertion of the particles and the motion of the particles. Thus, it is understood that the optical lens could be replaced by, or augmented by, another imaging apparatus (for example a small-scale single-sided MRI system). For the purpose of the disclosed embodiments, the term “imaging,” includes imaging technology that utilize components to form an image using magnetic resonance or magnetic particle imaging. It should be understood that such components include coils or magnets (or electro-permanent magnets) that polarize protons or other nuclei or electrons in one or more structures to be imaged, wherein gradient and/or radiofrequency coils may be used form an image of the structure. Thus, although not shown in detail herein, it should be understood that the disclosed embodiments may be used in conjunction with a support structure that may hold an imaging system and may contain other components needed to operate or move the imaging system, for example, wheels and/or batteries. Moreover, it should be understood that an associated display system is not shown but should be understood to be present in order to view images produced by the imaging system.

Disclosed embodiments of the apparatus may be operated to provide a method to disrupt biofilms of microbes 150 so as to increase the effectiveness of topical and/or systemic antimicrobial medications given to the patient in combating the infection 230. For the purposes of this disclosure, a biofilm may be defined as an assembly of microbes that is harder to treat than non-assembled microbes. Non-assembled microbes are sometimes described as “planktonic”.

Thus, in accordance with the disclosed embodiments, magnetic particles 300 are introduced into the subject's body part and moved in response to a magnetic field created by magnetic field generator 270. It is understood that the term “magnetic field generator” may include coils or permanent magnets or magnetizable or magnetoelectric materials for generating electromagnetic fields, electric fields, or magnetic fields. These fields may be used to collect an image (e.g. through MRI or Magnetic Particle Imaging (MPI)) and/or to manipulate the particles introduced into the subject's body.

Two important aspects of the disclosed embodiments include: (a) that the magnetic field produced by generator 270 can increase the transportation of particles 300 into the nailbed; and (b) that the motion of particles 300 under the influence of generator 270 can disrupt microbial biofilms and thereby increase the effectiveness of medications given to the subject. Such motion may include rotation, translation, vibration, or wobbling or a combination of those or other motions.

It is understood that magnetic generator 270 may include coils 280 and 290 of different orientations than are shown in FIG. 2 and enable movement of the particles in various manners. For example, published evidence that moving magnetic particles can increase the effectiveness of anti-fungal agents is provided by the scientific publication entitled “Biofilm disruption with rotating microrods enhances antimicrobial efficiency,” published by L. O. Mair et al. in the Journal of Magnetism and Magnetic Materials (incorporated herein by reference in its entirety), and disclosed in US patent application 20170227617, entitled “Method and Apparatus for Manipulating Electropermanent Magnets for Magnetic Resonance Imaging and Image-Guided Therapy” (incorporated herein by reference in its entirety).

It is understood that the administration of particles and disruption of biofilms could be done with optical guidance, as shown in FIG. 2, where optical objective lens 310 may be part of a larger optical microcopy or imaging device.

Optionally, particles 300 may contain or be coated with medications that will kill or weaken microbes 250. Thus, in accordance with disclosed embodiments, an apparatus and method may be used to propel drug-loaded or non-drug-loaded magnetic particles within a subject's body to a hard-to-access body part.

In accordance with at least one disclosed embodiment, the magnetic field generator 270 could constitute a MRI system for imaging, as in U.S. patent application Ser. No. 13/761,200, “Equipment and methodologies for magnetically-assisted delivery of therapeutic agents through barriers” by Irving Weinberg (incorporated herein by reference in its entirety), and application Ser. No. 15/427,426: “Method and apparatus for manipulating electropermanent magnets for magnetic resonance imaging and image guided therapy” by Irving Weinberg (incorporated herein by reference in its entirety).

In accordance with at least some embodiments, the MRI may be single-sided (i.e. disposed on one side of the body part) or may be more than one-side (e.g. two-sided). Alternatively the magnetic generator 270 may constitute a “magnetic particle imaging” system to detect the particles 300 and superimpose those particle images on an optical image of the nail bed. For the purpose of this disclosure the various methods of detecting and visualizing the particles is described as “magnetic means of tracking the particles”.

The technical effect of the disclosed embodiments provides the ability to improve the efficacy of drugs in changing the natural course of, for example, onychomycosis, by further enabling delivery of medication and otherwise altering the environment by which the infection spreads by manipulating particles to disrupt biofilm formation so as to increase the effectiveness of topical and/or systemic antimicrobial medications given to the patient in combating the infection or otherwise treat infected biofilm. Thus, a beneficial effect of using this apparatus is to deliver magnetic particles with or without medication into hard-to-access locations of a subject's body using real-time shaped dynamic magnetic fields.

Thus, in accordance with various embodiments, magnetic gradients and fields may be generated by the generator 270 both for propelling the magnetic particles through the subject's body part as well as, alternately, imaging and propelling the magnetic particles, as previously disclosed in above identified patent applications by Irving Weinberg. Thus, the generator 270 may include a system to apply magnetic fields under imaging guidance, wherein image-guidance components may include permanent magnets, electromagnets, antennas or electropermanent magnets.

Such electropermanent magnets may at one or more times create a magnetic field configuration for imaging of a subject's body part and then at another set of times create a magnetic field configuration for propulsion of particles. It should be understood that the imaging capability may be through magnetic resonance imaging methods. This may be performed using oscillating and/or static magnetic fields. The term “oscillating magnetic field” is defined as alternating magnetic fields provided by Helmholtz coils. For the purposes of this specification, the term “oscillating” means changing in direction and/or magnitude. The term “static magnetic field” is defined as a magnetic field which does not change in intensity or direction over time.

It should be understood that the terms “magnetic particles,” “nanoparticles,” and “magnetic nanoparticles,” may be defined as one or more particles made of material that exhibits magnetic or electric properties after or during exposure to a magnetic field. It should be understood that the term “particle” means an object smaller than 1 mm, 100 micron, 10 microns, 1 micron, 0.1 microns, or 0.01 microns in the smallest diameter and less than 1 mm, 100 microns, 10 microns, 1 micron, or 0.01 microns in the largest dimension.

The terms “near” and “proximity” may be less than one meter.

It is understood that the body part may be other than a toe, for example the subject's body part may be a fingernail, or other location in the body where infections may be difficult to treat.

As explained above with reference to FIG. 2, disclosed embodiments enable administration of particles and disruption of biofilms under optical guidance, as shown in FIG. 2, where optical objective lens 310 may be part of a larger optical microcopy or imaging device.

It should be understood that the disclosed apparatus and methodologies may be used in conjunction with other components, for example a computer and/or a power supply and/or coils for generating magnetic and/or electromagnetic fields, in order to attain a desired result of a meaningful image. It is understood that the image may use principles of proton magnetic resonance imaging, or magnetic resonance imaging of other particles (for example, electrons or sodium atoms) or other imaging principles (for example, magnetic particle imaging, or impedance imaging). It is understood that the apparatus may be used to deliver therapy by manipulating magnetizable materials with the magnetic field produced by the device. It should be understood that this manipulation may be performed at one time, and that imaging may be performed at another time, in order to guide the manipulation described above.

It should be understood that one or more magnetic fields applied by the generator 270 to a body part of a subject may be so rapidly applied so as not to cause unpleasant nerve stimulation, as taught by Irving Weinberg in issued U.S. Pat. No. 8,154,286, entitled “APPARATUS AND METHOD FOR DECREASING BIO-EFFECTS OF MAGNETIC FIELDS,” and related applications related through priority rights by Irving Weinberg, incorporated herein by reference.

It is understood that the apparatus may be further employed to magnetically remove particles from the subject's body part after having delivered therapy, and that the removal may be useful in order to reduce the potential of the subject's bodily reactions to the particles.

With the above description in mind, it should be understood that the term “subject” refers to and includes humans and other animals, whether they be alive or once-living. Similarly, the term “body part or other structure” may mean a tissue-containing structure in a living or once-living organism such as a human or other animal.

Likewise, it should be understood that the term “structure” may mean a tissue-containing structure in a living or once-living organism such as a human or other non-human animal.

It should be understood that the term “magnetizable” and “magnetic” are used interchangeably to indicate a material that can be magnetized.

It is understood that the action of the assemblies is under the control of a computer, and may be autonomously targeted to one or more targets on the basis of the magnetic resonance images of the body part and targets.

It should be understood that the operations explained herein may be implemented in conjunction with, or under the control of, one or more general purpose computers running software algorithms to provide the presently disclosed functionality and turning those computers into specific purpose computers.

Moreover, those skilled in the art will recognize, upon consideration of the above teachings, that the above exemplary embodiments may be based upon use of one or more programmed processors programmed with a suitable computer program. However, the disclosed embodiments could be implemented using hardware component equivalents such as special purpose hardware and/or dedicated processors. Similarly, general purpose computers, microprocessor based computers, micro-controllers, optical computers, analog computers, dedicated processors, application specific circuits and/or dedicated hard wired logic may be used to construct alternative equivalent embodiments.

Moreover, it should be understood that control and cooperation of the above-described components may be provided using software instructions that may be stored in a tangible, non-transitory storage device such as a non-transitory computer readable storage device storing instructions which, when executed on one or more programmed processors, carry out the above-described method operations and resulting functionality. In this case, the term non-transitory is intended to preclude transmitted signals and propagating waves, but not storage devices that are erasable or dependent upon power sources to retain information.

Those skilled in the art will appreciate, upon consideration of the above teachings, that the program operations and processes and associated data used to implement certain of the embodiments described above can be implemented using disc storage as well as other forms of storage devices including, but not limited to non-transitory storage media (where non-transitory is intended only to preclude propagating signals and not signals which are transitory in that they are erased by removal of power or explicit acts of erasure) such as for example Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments. Such alternative storage devices should be considered equivalents.

While certain illustrative embodiments have been described, it is evident that many alternatives, modifications, permutations and variations will become apparent to those skilled in the art in light of the foregoing description. Accordingly, the various embodiments of, as set forth above, are intended to be illustrative, not limiting. Various changes may be made without departing from the spirit and scope of the invention.

1. An apparatus for delivering one or more magnetic particles to a subject's body part to treat one or more infections in the subject's nail bed, the apparatus comprising:
a magnetic field generator including one or more coils disposed near to the subject's body part and outside the subject's body part and a controller coupled to the one or more coils and configured to control the one or more coils to generate an oscillating magnetic field;
at least one image-guidance component positioned in proximity to the subject's body part; and
an applicator for manipulating the one or more magnetic particles into and within the subject's body part using the oscillating magnetic field generated by the magnetic field generator,
wherein the at least one image-guidance component provides imaging data that enables delivery and manipulation of the one or more magnetic particles to the subject's body part.

2. The apparatus of claim 1, further comprising a plurality of magnetic particles including the one or more magnetic particles.

3. The apparatus of claim 2, wherein the plurality of magnetic particles contain or are coated with one or more anti-microbial substances.

4. The apparatus of claim 1, wherein the manipulation of the one or more particles is performed under imaging guidance to disrupt one or more biofilms in the subject's body part.

5. The apparatus of claim 1, wherein the at least one image-guidance component performs tracking of the one or more magnetic particles.

6. The apparatus of claim 1, wherein the tracking is optical tracking.

7. The apparatus of claim 1, wherein the tracking is magnetic imaging tracking.

8. The apparatus of claim 1, wherein the subject's body part is a nail bed.

9. The apparatus of claim 1, wherein the infection is at least in part from fungal organisms.

10. The apparatus of claim 1, wherein the at least one image-guidance component comprises an MRI system that includes the at least one magnetic coil.

11. The apparatus of claim 10, wherein the MRI system generates pulse sequences wherein the MRI has pulse sequences whose rise-time, fall-time, or duration are less than 10 microseconds long.

12. The apparatus of claim 10, wherein the MRI system includes at least one magnetic coil generates a magnetic field that rises or falls in such short a time as not to cause nerve stimulation of the subject.

13. A method for delivering one or more magnetic particles to a subject's body part to treat one or more infections in the subject's nail bed, the methods comprising:
generating an oscillating magnetic field using a magnetic field generator including one or more coils disposed near to the subject's body part and outside the subject's body part and a controller coupled to the one or more coils and configured to control the one or more coils to generate the oscillating magnetic field;
imaging the subject's body part using at least one image-guidance component positioned in proximity to the subject's body part; and
manipulating the one or more magnetic particles using an applicator to manipulate the one or more magnetic particles into and within the subject's body part using the oscillating magnetic field generated by the magnetic field generator,
wherein the at least one image-guidance component provides imaging data that enables delivery and manipulation of the one or more magnetic particles to the subject's body part.

14. The method of claim 13, wherein the one or more magnetic particles are included in a plurality of magnetic particles delivered and manipulated by the method.

15. The method of claim 14, wherein the plurality of magnetic particles contain or are coated with one or more anti-microbial substances.

16. The method of claim 13, wherein the manipulation of the one or more particles is performed under imaging guidance to disrupt one or more biofilms in the subject's body part.

17. The method of claim 13, wherein the at least one image-guidance component performs tracking of the one or more magnetic particles.

18. The method of claim 13, wherein the tracking is optical tracking.

19. The method of claim 13, wherein the tracking is magnetic imaging tracking.

20. The method of claim 13, wherein the subject's body part is a nail bed.

21. The method of claim 13, wherein the infection is at least in part from fungal organisms.

22. The method of claim 13, wherein the at least one image-guidance component comprises an MRI system that includes the at least one magnetic coil.

23. The method of claim 22, wherein the MRI system generates pulse sequences wherein the MRI has pulse sequences whose rise-time, fall-time, or duration are less than 10 microseconds long.

24. The method of claim 22, wherein the MRI system includes at least one magnetic coil generates a magnetic field that rises or falls in such short a time as not to cause nerve stimulation of the subject.
